SEQUENCE LISTING (27 sequences)

SEQ ID NO: 1 - protein, 7 aa, Artificial Sequence
  Synthetic peptide
LPLGNSH

SEQ ID NO: 2 - protein, 7 aa, Artificial Sequence
  Synthetic peptide
SWWAPFH

SEQ ID NO: 3 - protein, 7 aa, Artificial Sequence
  Synthetic peptide
YPVHPST

SEQ ID NO: 4 - protein, 7 aa, Artificial Sequence
  Synthetic peptide
PILQAGL

SEQ ID NO: 5 - protein, 28 aa, Artificial Sequence
  Synthetic peptide
GLRSKSKKFRRPDIQYPDATDEDITSHM

SEQ ID NO: 6 - protein, 37 aa, Artificial Sequence
  Synthetic peptide
FNKHTEIIEEDTNKDKPSYQFGGHNSVDFEEDTLPKV

SEQ ID NO: 7 - protein, 12 aa, Artificial Sequence
  Synthetic peptide
GAHWQFNALTVR

SEQ ID NO: 8 - protein, 13 aa, Artificial Sequence
  Synthetic peptide
GKKQRFRHRNRKG

SEQ ID NO: 9 - protein, 7 aa, Artificial Sequence
  Synthetic peptide
NNHYLPR

SEQ ID NO: 10 - protein, 5 aa, Artificial Sequence
  Synthetic peptide
GGGGS

SEQ ID NO: 11 - protein, 15 aa, Artificial Sequence
  Synthetic peptide
EAAAKEAAAKEAAAK

SEQ ID NO: 12 - protein, 5 aa, Artificial Sequence
  Synthetic peptide
EAAAK

SEQ ID NO: 13 - protein, 46 aa, Artificial Sequence
  Synthetic peptide
AEAAAKEAAAKEAAAKEAAAKALEAEAAAKEAAAKEAAAKEAAAKA

SEQ ID NO: 14 - protein, 5 aa, Artificial Sequence
  Synthetic peptide
PAPAP

SEQ ID NO: 15 - protein, 12 aa, Artificial Sequence
  Synthetic peptide
AEAAAKEAAAKA

SEQ ID NO: 16 - protein, 5 aa, Artificial Sequence
  Synthetic peptide
GGGGS

SEQ ID NO: 17 - protein, 17 aa, Artificial Sequence
  Synthetic peptide
VSQTSKLTRAETVFPDV

SEQ ID NO: 18 - protein, 6 aa, Artificial Sequence
  Synthetic peptide
PLGLWA

SEQ ID NO: 19 - protein, 6 aa, Artificial Sequence
  Synthetic peptide
RVLAEA

SEQ ID NO: 20 - protein, 18 aa, Artificial Sequence
  Synthetic peptide
EDVVCCSMSYGGIEGRGS

SEQ ID NO: 21 - protein, 10 aa, Artificial Sequence
  Synthetic peptide
TRHRQPRGWE

SEQ ID NO: 22 - protein, 10 aa, Artificial Sequence
  Synthetic peptide
AGNRVRRSVG

SEQ ID NO: 23 - protein, 9 aa, Artificial Sequence
  Synthetic peptide
RRRRRRRRR

SEQ ID NO: 24 - protein, 4 aa, Artificial Sequence
  Synthetic peptide
GFLG

SEQ ID NO: 25 - protein, 25 aa, Artificial Sequence
  Synthetic peptide
RLVFALGTDGKKLRIKSKEKCNDGK

SEQ ID NO: 26 - protein, 10 aa, Artificial Sequence
  Synthetic peptide
EDVVCCSMSY

SEQ ID NO: 27 - protein, 31 aa, Artificial Sequence
  Synthetic peptide
GGGGLRSKSKKFRRPDIQYPDATDEDITSHM

(19) United States
(12) Patent Application Publication
(10) Pub. No.: US 2023/0312685 A1
(43) Pub. Date: Oct. 5, 2023

(54) Bi-Peptide with Affinity to Extracellular Matrix Proteins or Cells and to Growth Factors for Tissue Healing and Regeneration

(71) Applicant: Mayo Foundation for Medical Education and Research, Rochester (US)

(72) Inventors: Joao Francisco Crispim, Odemira (PT); Daniel B. Saris, Rochester, MN (US)

(21) Appl. No.: 17/768,537

(22) PCT Filed: Oct. 23, 2020
(86) PCT No.: PCT/US2020/057090

§ 371 Date: Apr. 13, 2022

Related U.S. Application Data
(60) Provisional application No. 62/926,180, filed on Oct. 25, 2019.

(51) Int. Cl. C07K 14/78; C07K 14/71; A61L 27/22; A61L 27/54

(52) U.S. Cl. CPC C07K 14/78; C07K 14/71; A61L 27/227; A61L 27/54; C07K 2319/33; A61K 38/00

(57) ABSTRACT

The present invention includes a bi-peptide, a method of making, and a method using the bi-peptide to treat a disease or condition, wherein the bi-peptide comprises the formula: peptide 1ₙ-linkerₙ-peptide 2ₙ, or peptide 2ₙ-linkerₙ-peptide 1ₙ wherein peptide 1 has an affinity to a growth factor or a growth factor receptor, wherein peptide 2 has an affinity for an extracellular matrix protein, and wherein the linker is a chemical or peptide linker, and n is one or more.

### CROSS-REFERENCE TO RELATED APPLICATIONS

This application claims priority to U.S. Provisional Application Ser. No. 62/926,180, filed Oct. 25, 2019, the entire contents of which are incorporated herein by reference.

### TECHNICAL FIELD OF THE INVENTION

The present invention relates in general to the field of peptides and growth factors, and more particularly, to a bi-peptide with affinity to extracellular matrix proteins, cells, and growth factors for tissue healing and regeneration.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Oct. 23, 2020, is named MAYO2010WO_SeqList.txt and is 6, kilobytes in size.

### BACKGROUND OF THE INVENTION

Without limiting the scope of the invention, its background is described in connection with tissue repair.

Owing to an aging population and involvement in physical activities, musculoskeletal injuries are among the most common injures worldwide. From about 33 million injuries reported in the US per year, approximately 33% involve T/L (James et al., 2008. J Hand Surg Am 33: 102-12). The most frequently reported injury is that of the anterior cruciate ligament (ACL) tear or rupture, which accounts for more than 80,000 cases per year in the US alone, with an estimated cost of US$1.0 billion (Griffin et al., 2000. J Am Acad Orthop Surg 8: 141-50). Due to improvements in quality of life and the increasing participation of the population in physical activities, the incidence of these injuries is likely to rise, affecting patient quality of life and increasing healthcare costs. Injuries to these tissues are always associated with pain, swelling and disability, with the extent of the damage dictating recovery time (Chen et al, 2008. Curr Rev Musculoskelet Med 1: 108-113; Medicine 2016; Knee Ligament Repair).

When tears or ruptures of the tissues occur, surgical intervention is usually needed. The main goal of this surgical treatment is to stabilize and restore normal movement to the joint (Baumhauer and O'Brien, 2002. J Athl Train 37: 458-462). Due to the nature of these tissues and their inherent poor healing capacity, surgical intervention is also needed to direct the natural healing process. However, even with the available treatments, complete healing of the damaged tissue is difficult to achieve, which can ultimately lead to scarring, restrictions to the range of motion, stiffness/weakness of the joint, improper healing and re-injury (Krans, 2016. ACL Reconstruction. Available from healthline.com/health/acl-reconstruction#Overviewl). After surgery, a long recovery period is required of between 9 to 12 months, during which the patient's movement and quality of life are affected and the pre-injury properties of the T/L are likely not yet fully restored (Voleti et al., 2012. Annu Rev Biomed Eng, 14: 47-71).

Consequently, there is a need to improve the current treatments and make the overall surgical and rehabilitation process more efficient, shorter and friendlier to the patient, not only for treatment of tendons and ligaments, but for treatment of a tissue injury in general.

### SUMMARY OF THE INVENTION

In one embodiment, the present invention includes a bi-peptide comprising a formula: peptide 1ₙ-linkerₙ-peptide 2ₙ, or peptide 2ₙ-linkerₙ-peptide 1ₙ, wherein peptide 1 has an affinity to a growth factor or a growth factor receptor, wherein peptide 2 has an affinity for an extracellular matrix protein, and wherein the linker is a chemical or peptide linker; and n is one or more. In one aspect, peptide 1 is selected from a TGF-B1, VEGF, BMP-2, PDGF, or an FGF binding peptide. In another aspect, peptide 2 is selected from a collagen, fibronectin, hyaluronan, hydroxyapatite, or a heparin binding peptide. In another aspect, peptide 1 is selected from LPLGNSH (SEQ ID NO:1), SWWAPFH (SEQ ID NO:2), YPVHPST (SEQ ID NO:3), or a PILQAGL (SEQ ID NO:4) peptide. In another aspect, peptide 2 is selected from GLRSKSKKFRRPDIQYPDATDEDITSHM (SEQ ID NO:5), FNKHTEIIEEDTNKDKPSYQFGGHNSVDFEEDTLPKV (SEQ ID NO:6), GAHWQFNALTVR (SEQ ID NO:7), GKKQRFRHRNRKG (SEQ ID NO:8), NNHYLPR (SEQ ID NO: 9), or RLVFALGTDGKKLRIKSKEKCNDGK (SEQ ID NO: 25) peptide. In another aspect, further comprising the bi-peptide is bound to a polymer. In another aspect, the bi-peptide further comprises a second linker attached to at least one of peptide 1, peptide 2, or both, opposite the linker, and one or more additional peptide 1, peptide, or both peptide 1 and peptide 2 attached to the second linker. In another aspect, the bi-peptide further comprises concatamers of peptide 1, peptide 2, or both attached to peptide 1, peptide 2, or both. In another aspect, the linker is selected from a small molecule, a peptide, a nucleic acid, a carbohydrate, or a lipid.

In another embodiment, the present invention includes a device comprising the bi-peptide.

In another embodiment, the present invention includes a bi-peptide or the device of claim 10, for use in the treatment of a tissue pathology or tissue engineering, preferably a tissue injury. In one embodiment, the present invention includes a bi-peptide for use in the treatment of an injured tendon and/or ligament.

In another embodiment, the present invention includes a method for treatment of a tissue pathology in a subject, the method comprising: (a) providing a bi-peptide comprising a formula:

peptide 1ₙ-linkerₙ-peptide 2ₙ, or

peptide 2ₙ-linkerₙ-peptide 1ₙ

wherein peptide 1 has an affinity to a growth factor or a growth factor receptor, wherein peptide 2 has an affinity for an extracellular matrix protein, wherein the linker is a chemical or peptide linker, and n is one or more; (b) introducing the bi-peptide or device into tissue of the subject; and (c) allowing the bi-peptide or device to capture growth factor from the subject. In one aspect, the method further comprises mixing or attaching the bi-peptide to a biopolymer, wherein the biopolymer is selected from the group consisting of collagen, chitosan, dextran, hyaluronic acid, heparin, polysaccharides such as alginate, hyaluronic acid and agarose, polynucleotides, polypeptides, starch, polylactic acid, poly-L-lactic acid, polyglycolic acid, polyglycolic lactic acid, poly(amidoamine), poly(caprolactone), polyalkyleneoxide-polyalkylene-terephtalate block copolymer, poly-N-isopropylacrylamide, polyurethane, poly-acrylate, polyesters, polystyrene, polycarbonate, polyethyleneterephtalate (PET) polybutyleneterephtalate (PBT), polyethyleneoxide (PEO), polyethersulfone (PES), polytetrafluoroethylen (PTFE), polytrimethylenecaprolactone (PTMC), polyanhydride, poly(ortho)ester, polyphosphazene, and/or combinations thereof. In another aspect, the tissue injury is an injured tendon and/or ligament. In another aspect, peptide 1 is selected from a TGF-B1, VEGF, BMP-2, or an FGF binding peptide. In another aspect, peptide 2 is selected from a collagen, fibronectin, hyaluronan, hydroxyapatite or a heparin binding peptide. In another aspect, peptide 1 is selected from LPLGNSH (SEQ ID NO:1), SWWAPFH (SEQ ID NO:2), YPVHPST (SEQ ID NO:3), or a PILQAGL (SEQ ID NO:4) peptide. In another aspect, peptide 2 is selected from GLRSKSKKFRRPDIQYPDATDEDITSHM (SEQ ID NO:5), FNKHTEIIEEDTNKDKPSYQFGGHNSVDFEEDTLPKV (SEQ ID NO:6), GAHWQFNALTVR (SEQ ID NO:7), GKKQRFRHRNRKG (SEQ ID NO:8), NNHYLPR (SEQ ID NO: 9), or RLVFALGTDGKKLRIKSKEKCNDGK (SEQ ID NO: 25) peptide. In another aspect, the method further comprises binding the bi-peptide to a polymer. In another aspect, the method further comprises adding a second linker attached to at least one of peptide 1, peptide 2, or both, opposite the linker, and one or more additional peptide 1, peptide, or both peptide 1 and peptide 2 attached to the second linker. In another aspect, the method further comprises concatamers of peptide 1, peptide 2, or both attached to peptide 1, peptide 2, or both. In another aspect, the method further comprises the linker is selected from a small molecule, a peptide, a nucleic acid, a carbohydrate, or a lipid.

In another embodiment, the present invention includes a method of making a bi-peptide comprising a formula:

peptide 1ₙ-linkerₙ-peptide 2ₙ, or

peptide 2ₙ-linkerₙ-peptide 1ₙ

obtaining a peptide 1 that has an affinity to a growth factor or a growth factor receptor; connecting a linker to peptide 1, wherein the linker is a chemical or peptide 1; and connecting a peptide 2 has an affinity for an extracellular matrix protein to the linker, wherein n is one or more. In one aspect, the method further comprises selecting peptide 1 from a TGF-B1, VEGF, BMP-2, or an FGF binding peptide. In another aspect, the method further comprises selecting peptide 2 from a collagen, fibronectin, hyaluronan, hydroxyapatite, or a heparin binding peptide. In another aspect, the method further comprises selecting peptide 1 from LPLGNSH (SEQ ID NO:1), SWWAPFH (SEQ ID NO:2), YPVHPST (SEQ ID NO:3), or a PILQAGL (SEQ ID NO:4) peptide. In another aspect, the method further comprises selecting peptide 2 from GLRSKSKKFRRPDIQYPDATDEDITSHM (SEQ ID NO:5), FNKHTEIIEEDTNKDKPSYQFGGHNSVDFEEDTLPKV (SEQ ID NO:6), GAHWQFNALTVR (SEQ ID NO:7), GKKQRFRHRNRKG (SEQ ID NO:8), NNHYLPR (SEQ ID NO: 9), or RLVFALGTDGKKLRIKSKEKCNDGK (SEQ ID NO: 25) peptide. In another aspect, the method further comprises binding the bi-peptide to a polymer. In another aspect, the method further comprises attaching a second linker attached to at least one of peptide 1, peptide 2, or both, opposite the linker, and one or more additional peptide 1, peptide, or both peptide 1 and peptide 2 attached to the second linker.

### BRIEF DESCRIPTION OF THE DRAWINGS

For a more complete understanding of the features and advantages of the present invention, reference is now made to the detailed description of the invention along with the accompanying figures and in which:

FIG. 1 shows images of the incubation of the fluorescently labelled collagen-binding motif with the collagen meniscal implant (CMI). Note: the CMI is build out of collagen type 1 from bovine origin (FDA approved implant). 2D imaging with fluorescence microscope.

FIG. 2 shows images of the incubation of the fluorescently labelled collagen-binding motif with human meniscal allograft tissue. 2D imaging with fluorescence microscope.

FIG. 3 shows images of the incubation of the fluorescently labelled collagen-binding motif with human clinical-grade fresh frozen hamstring tendon allograft. 2D imaging with fluorescence microscope.

FIG. 4 shows images of the incubation of the fluorescently labelled collagen-binding motif with human clinical-grade fresh frozen patellar tendon allograft. 2D imaging with fluorescence microscope.

FIG. 5 is a graph that shows mean fluorescence intensity for each collagen-containing tissue after incubation with the fluorescently labelled collagen binding motif peptide.

FIG. 6 shows images of an immunofluorescence assay for VEGF 125-a captured by the bi-peptide structure on the CMI. Pictures are merged z-stack files from confocal microscopy.

FIG. 7 is a graph that shows mean fluorescence intensity for the IF assay capturing VEGF. (1) Complete assay; (2) Blank assay; (3) Assay without peptide; (4) Assay without growth factor (VEGF); and (5) Assay without primary antibody.

FIG. 8 shows images of an immunofluorescence assay for PDGF-BB captured by the bi-peptide structure on the CMI. Pictures are merged z-stack files from confocal microscopy.

FIG. 9 is a graph that shows mean fluorescence intensity for the IF assay capturing PDGF. (1) Complete assay; (2) Blank assay; (3) Assay without peptide; (4) Assay without growth factor (PDGF); (5) Assay without primary antibody.

FIG. 10 is an image of a Tile confocal scan of the CMI in the IF assay for PDGF-BB. The complete surface of the CMI was able to bind with recombinant PDGF.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “growth factor” refers to a molecule that elicits a biological response to improve tissue regeneration, tissue growth and/or organ function. Preferred growth factors are morphogens. The term ‘morphogen’ as used herein refers to a substance governing the pattern of tissue development and, preferably, the positions of the various specialized cell types within a tissue. A morphogen spreads from a localized source and forms a concentration gradient across a developing tissue. The growth factor may be selected from the group consisting of platelet derived growth factor (PDGF) AA, PDGF BB, insulin-like growth factors, fibroblast growth factors (FGF), β-endothelial cell growth factor, transforming growth factors (TGF), such as TGFβ1 (TGFβ1), TGFβ2, TGFβ3, TGFβ5; bone morphogenic protein (BMP) 1, BMP2, BMP 3, BMP 4, BMP 7, vascular endothelial growth factor (VEGF), placenta growth factor; epidermal growth factor (EGF), amphiregulin, betacellulin, heparin binding EGF, Interleukins (IL), such as, IL-1, IL-2, IL-3, IL-4, IL-5, IL-6, IL-7, IL-8, IL-9, IL-10, IL-11, IL-12, IL-13, IL-14, IL-15-18, colony stimulating factor (CSF)-G, CSF-GM, CSF-M, erythropoietin, nerve growth factor (NGF), ciliary neurotropic factor, stem cell factor, and hepatocyte growth factor. The term growth factor, as used herein, includes naturally occurring growth factor receptor antagonists such as angiopoietin-2 and fetuin.

A growth factor belongs to the TGF beta superfamily, including the TGF beta subfamily, the decapentaplegic Vg-related (DVR) related subfamily, and the activin and inhibin subfamily. A preferred growth factor of the TGF beta superfamily is selected from the group consisting of anti-Müllerian hormone, artemin, BMP2, BMP3, BMP4, BMP5, BMP6, BMP7, BMP8A, BMP8B, BMP10, BMP 15, growth differentiation factor-1 (GDF1), GDF10, GDF11, GDF15, GDF2, GDF3, GDF3A, GDF5, GDF6, GDF7, GDF8, GDF9, glial cell-derived neurotrophic factor, inhibin alpha, inhibin beta A, inhibin beta B, inhibin beta C, inhibin beta E, left-right determination factor 1, left-right determination factor 2, myostatin, NODAL, neurturin, persephim, TGFB1, TGFB2, TGFB3 and TGFB5.

The term growth factor binding peptide, as is used herein, refers to a peptide that is able to bind with high affinity to a growth factor, preferably a human growth factor. The binding peptide preferably binds to a growth factor while allowing the growth factor to elicit its biological function to improve tissue regeneration, tissue growth and/or organ function. The binding peptide preferably binds specific to a growth factor. The term ‘specific’ or grammatical variations thereof refers to the number of different types of growth factors, or their epitopes, to which a particular peptide can bind. The specificity of an peptide can be determined based on affinity. The affinity of a peptide for its target is a quality defined by a dissociation constant (KD). Preferably the KD is less than 10⁻⁵, less than 10⁻⁶, less than 10⁻⁷, less than 10⁻⁸, less than 10⁻⁹ and even less than 10⁻¹⁰ molar. Methods of determining affinity are known in the art, for example as described in Gilson and Zhou, 2007 (Gilson and Zhou, 2007. Annual Review of Biophysics and Biomolecular Structure 36: 21-42).

A growth factor binding peptide preferably binds to a mammalian growth factor, more preferably a human growth factor.

The current invention includes a method to target cells and the extracellular matrix (ECM) and at the same time capture and deliver growth factors.

This is achieved by developing a bi-peptide that includes two peptides connected by a linker. One peptide displays affinity towards a component of the extracellular matrix, such as collagen, hyaluronic acid, fibronectin or any other matrix protein. The second peptide displays affinity towards growth factors that have a role in tissue healing such as transforming growth factor beta, bone morphogenetic protein, vascular endothelial growth factor or any other growth factor involved in tissue repair. In one example, the bi-peptide has the following structure:

peptide 1ₙ-linkerₙ-peptide 2ₙ, or

peptide 2ₙ-linkerₙ-peptide 1ₙ

wherein peptide 1 has an affinity to a growth factor or a growth factor receptor, wherein peptide 2 has an affinity for an extracellular matrix protein, wherein the linker is a chemical or peptide linker, and n is one or more.

These bi-peptides can be used in methods that allow for specific targeting and binding to tissues through the affinity of the ECM binding peptide to ECM proteins, and capture of endogenous growth factors through the affinity of the growth factor binding peptide to the respective growth factor.

The bi-peptide consists of two peptide sequences connected together through a linker such as Polyethylene Glycol or other linkers known in the art. Non-limiting examples of linker include sequences for growth factors and ECM proteins, which may either be amino or carboxy, in other words, the peptides can be Peptide 1ₙ-Linker-Peptide 2ₙ; or Peptide 2ₙ-Linker-Peptide 1ₙ; but may also include multiple linkers (Linkerₙ), and multiple peptides on either the amino or carboxy ends, or combinations of peptides. Examples of peptides for use with the present invention include one or more of the following:

Growth Factor peptides (Peptide 1):

TGF-B1
(SEQ ID NO: 1)
LPLGNSH
VEGF
(SEQ ID NO: 2)
SWWAPFH
BMP-2
(SEQ ID NO: 3)
YPVHPST
FGF
(SEQ ID NO: 4)
PILQAGL

ECM proteins peptides (Peptide 2):

Collagen
(SEQ ID NO: 5)
GLRSKSKKFRRPDIQYPDATDEDITSHM
Fibronectin
(SEQ ID NO: 6)
FNKHTEIIEEDTNKDKPSYQFGGHNSVDFEEDTLPKV
Hyaluronan
(SEQ ID NO: 7)
GAHWQFNALTVR
Heparin
(SEQ ID NO: 8)
GKKQRFRHRNRKG
Hydroxyapatite
(SEQ ID NO: 9)
NNHYLPR

Linkers for use with the present invention include, e.g., peptides, small molecules, nucleic acids, carbohydrates, or lipids. In one embodiment the bifunctional chemical linker is heterobifunctional linker. Suitable heterobifunctional chemical linkers include polyethylene glycol, N-succinimidyl 3-(2-pyridyldithio)propionate (SPDP) or N,N′-(1,3-phenylene) bismaleimide; N,N′-ethylene-bis-(iodoacetamide) or other such reagent having 6 to 11 carbon methylene bridges; and 1,5-difluoro-2,4-dinitrobenzene. Other cross-linking reagents useful for this purpose include, but are not limited to: iminothiolane (IT), bifunctional derivatives of imidoesters, active esters, aldehydes, bis-azido compounds, bis-diazonium derivatives, diisocyanates, bis-active fluorine compounds, p,p′-difluoro-m,m′-dinitrodiphenylsulfone; dimethyl adipimidate; phenol-1,4-disulfonylchloride; hexamethylenediisocyanate or diisothiocyanate, or azophenyl-p-diisocyanate; glutaraldehyde and disdiazobenzidine and any combination thereof. Cross-linking reagents may also be homobifunctional, i.e., having two functional groups that undergo the same reaction. A preferred homobifunctional cross-linking reagent is bismaleimidohexane (BMH). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of proteins (or polypeptides) that contain cysteine residues. Cross-linking reagents may also be heterobifunctional. Heterobifunctional cross-linking agents have two different functional groups, for example an amine-reactive group and a thiol-reactive group, that will cross-link two proteins having free amines and thiols, respectively. Nonlimiting examples of heterobifunctional cross-linking agents are Succinimidyl-4-(N-maleimidomethyl)-cyclohexane-1-carboxylate (SMCC), m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue. Because cross-linking reagents often have low solubility in water, a hydrophilic moiety, such as a sulfonate group, may be added to the cross-linking reagent to improve its water solubility. Sulfo-MBS and sulfo-SMCC are examples of cross-linking reagents modified for water solubility. Many cross-linking reagents yield a conjugate that is essentially non-cleavable under cellular conditions, which would not be preferred. Therefore, some cross-linking reagents contain a covalent bond, such as a disulfide, that is cleavable under cellular conditions. For example, Traut's reagent, dithiobis(succinimidylpropionate) (DSP), and N-succinimidyl 3-(2-pyridyldithio)propionate (SPDP) are well-known cleavable cross-linkers. The use of a cleavable cross-linking reagent permits the cargo moiety, e.g., Peptide 1 and Peptide 2, to separate from the linker after delivery into the target cell. For this purpose, direct disulfide linkage may also be useful. Chemical cross-linking may also include the use of spacer arms. Spacer arms provide intramolecular flexibility or adjust intramolecular distances between conjugated moieties and thereby may help preserve biological activity. A spacer arm may be in the form of a protein (or polypeptide) moiety that includes spacer amino acids, e.g., proline. Alternatively, a spacer arm may be part of the cross-linking reagent, such as in “long-chain SPDP” (e.g., Pierce Chem. Co., Rockford, Ill., cat. No. 21651 H). Numerous cross-linking reagents, including the ones discussed above, are commercially available. Detailed instructions for their use are readily available from the commercial suppliers. A general reference on protein cross-linking and conjugate preparation is Jameson and Wong, Chemistry of Protein Conjugation and Cross-Linking, CRC Press (1991), and Chen, et al., Fusion Protein Linkers: Property, Design and Functionality, Adv Drug Deliv Rev. 2013 Oct. 15; 65(10): 1357-1369, relevant portions incorporated herein by reference. Linkers can also be peptides, for example, a helical linker, a glyₙ, or a gly-serₙ linker. Amino acid sequences useful as include those disclosed in Maratea et al. (1985) Gene 40:39 46; Murphy et al. (1986) Proc. Natl. Acad. Sci. USA 83:8258 8262; and in U.S. Pat. Nos. 4,935,233 and 4,751,180, relevant portions and sequences incorporated herein by reference. The linker sequence may generally be from 1 to about 20 amino acids in length and may also include D- and/or L-amino acids.

As used herein, the terms “binding”, “specific binding” or “specifically binds to” or is “specific for” refers to the binding of a peptide to a binding target, such as the binding of a peptide that binds to a growth factor or its receptor, or a peptide that binds an extracellular matrix protein.

As used herein, the term “Binding affinity” refers to the strength of the sum total of noncovalent interactions between a single binding site of a molecule (e.g., a peptide) and its binding partner (e.g., a target), e.g., such as the binding of a peptide that binds to a growth factor or its receptor, or a peptide that binds an extracellular matrix protein.

TABLE 1
Examples of linkers include:

| Peptide Linker | SEQ ID NO: |
|---|---|
| (GGGGS)ₙ (n = 1-9) | 10 |
| (Gly)₈ | |
| (Gly)₆ | |
| (EAAAK)₃ | 11 |
| (EAAAK)ₙ (n = 1-9) | 12 |
| A(EAAAK)₄ALEA(EAAAK)₄A | 13 |
| PAPAP | 14 |
| AEAAAKEAAAKA | 15 |
| (GGGGS)ₙ (n = 1, 2, 4) | 16 |
| (Ala-Pro)ₙ (10-34 aa) | |
| disulfide | |
| VSQTSKLTR↓AETVFPDVD | 17 |
| PLG↓LWA | 18 |
| RVL↓AEA | 19 |
| GGIEGR↓GS | 20 |
| TRHRQPR↓GWE | 21 |
| AGNRVRR↓SVG | 22 |
| RRRRRRR↓R↓R^{d} | 23 |
| GFLG↓^{e} | 24 |
| EDVVCC↓SMSY | 26 |

Protease sensitive cleavage sites are indicated with “↓”

By injecting the bi-peptides it is possible to selectively target tissues and or biomaterials/matrices composed of ECM proteins and provide them with the ability to spatially control the capture endogenous growth factors. The capture and release to the surroundings of those growth factor will enhance the healing response of the target cells/tissue and lead to a stronger, better and faster regeneration of the damaged tissue.

### Example 1. Bi-Peptide Structure for Capturing Endogenous Growth Factors in Tissue Regeneration

The availability of growth factors at the site of injury is essential for tissue regeneration in any part of the body. Stem cells, immune cells and tissue specific host cells need the stimulus of growth factors to differentiate, to deposit newly formed extracellular matrix and to enhance nutrient supply by building new blood vessels leading to the injured zone. Growth factors identified to be involved in meniscal repair include PDGF, VEGF, FGF, CTGF and TGF-β and have shown to contribute to meniscal cell proliferation, increased collagen matrix production and meniscal cell migration in in-vitro studies under supra-physiological concentrations. Adding these exogenous recombinant growth factors in-vivo would lead to a short-lived boost of cellular migration and activity but its effect is easily nullified by removal from the knee joint and is most importantly very expensive. Therefore, the rationale of this study is to develop a sustainable long-lasting spatial availability of meniscal tissue specific growth factors from the body starting from the collagen meniscal implant (CMI®) scaffold as a frame for biological tissue repair. A bifunctional peptide will be synthesized that has the capacity to bind with collagen on one side to ensure proper attachment with the CMI. This amino acid sequence will be linked to another peptide that is able to bind and present the most predominant growth factors for meniscus repair to surrounding cells and stimulates cell adhesion at the same time. In this way, it is hypothesized to have an instantly and long-lasting higher than average concentration of bioactive endogenous growth factors at the repair site accelerating newly formed meniscal tissue.

The primary study aim was to design a bifunctional peptide sequence which contains a collagen-binding site at one end and a growth factor-binding site at the other end which can be attached to the CMI to promote meniscus regeneration. Secondary, the study wants to test the feasibility of tendon and bone auto/allograft functionalization with the bi-peptide structure according to the same biological strategy by targeting tissue-specific endogenous growth factors.
1. Binding confirmation
   a. Collagen binding site
      i. Peptide labelling with fluorescent dye
      ii. Incubation with several collagen containing implants/grafts
   b. Growth factor binding site
      i. Heparin binding domain (VEGF+PDGF)
         1. Under investigation
         2. Immunofluorescent assay on CMI
      ii. VEGF domain specific
         1. To be synthesized
      ii. BMP-2 domain specific
         1. To be synthesized
      iv. TGF-β1 domain specific
         1. To be synthesized
2. In-vitro study
3. Animal model

Current peptide amino acid sequences:

Heparin binding domain for VEGF-a125-linker-PDGF-BB

(SEQ ID NO: 5)
GLRSKSKKFRRPDIQYPDATDEDITSHM-
two 6 amino-hexanoic acid spacers-
(SEQ ID NO: 25)
RLVFALGTDGKKLRIKSKEKCNDGK

Application: tissue revascularization in meniscus, tendon or in poor wound healing

VEGF capturing:

(SEQ ID NO: 5)
GLRSKSKKFRRPDIQYPDATDEDITSHM-
two 6 amino-hexanoic acid spacers-
(SEQ ID NO: 2)
SWWAPFH

Application: revascularization and inflammatory protection in auto/allograft tissue (meniscus, ACL reconstruction)

BMP-2 capturing:

(SEQ ID NO: 3)
GLRSKSKKFRRPDIQYPDATDEDITSHM-
two 6 amino-hexanoic acid spacers-
(SEQ ID NO: 5)
YPVHPST

Application: bone allograft, tendon-bone interface healing in ACL reconstruction, non-union fractures

TGF-β1 capturing:

(SEQ ID NO: 5)
GLRSKSKKFRRPDIQYPDATDEDITSHM-
two 6 amino-hexanoic acid spacers-
(SEQ ID NO: 1)
LPLGNSH

Application: tendon and ligament regeneration, protection for initial inflammation and necrosis in allo/autograft tissue

All peptides: N-terminus: Free NH2, C-terminus: Acid (COOH).

Confirmation of collagen-binding motif to several collagen type 1 containing tissues.

Peptide sequence:
(SEQ ID NO: 27)
CF-GGG-GLRSKSKKFRRPDIQYPDATDEDITSHM

N-terminus: CF=carboxyfluorescein (fluorescent dye), C-terminus: Acid (COOH).

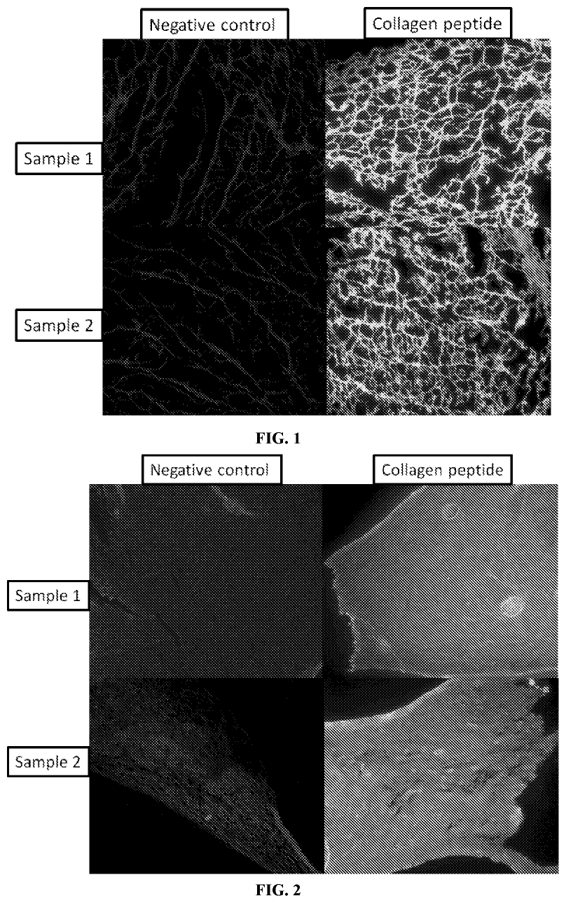

FIG. 1 shows images of the incubation of the fluorescently labelled collagen-binding motif with the collagen meniscal implant (CMI). Note: the CMI is build out of collagen type 1 from bovine origin (FDA approved implant). 2D imaging with fluorescence microscope.

FIG. 2 shows images of the incubation of the fluorescently labelled collagen-binding motif with human meniscal allograft tissue. 2D imaging with fluorescence microscope.

FIG. 3 shows images of the incubation of the fluorescently labelled collagen-binding motif with human clinical-grade fresh frozen hamstring tendon allograft. 2D imaging with fluorescence microscope.

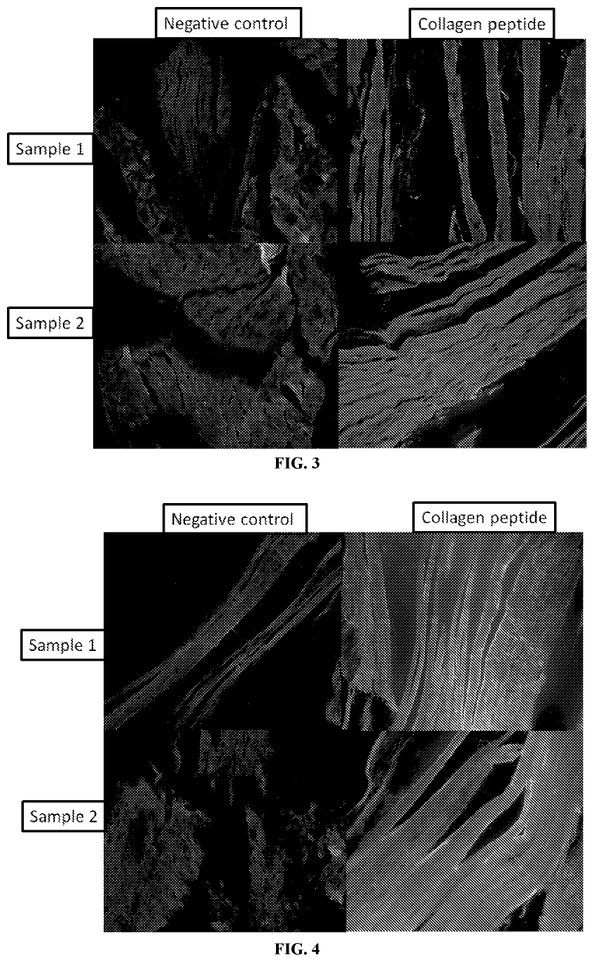

FIG. 4 shows images of the incubation of the fluorescently labelled collagen-binding motif with human clinical-grade fresh frozen patellar tendon allograft. 2D imaging with fluorescence microscope.

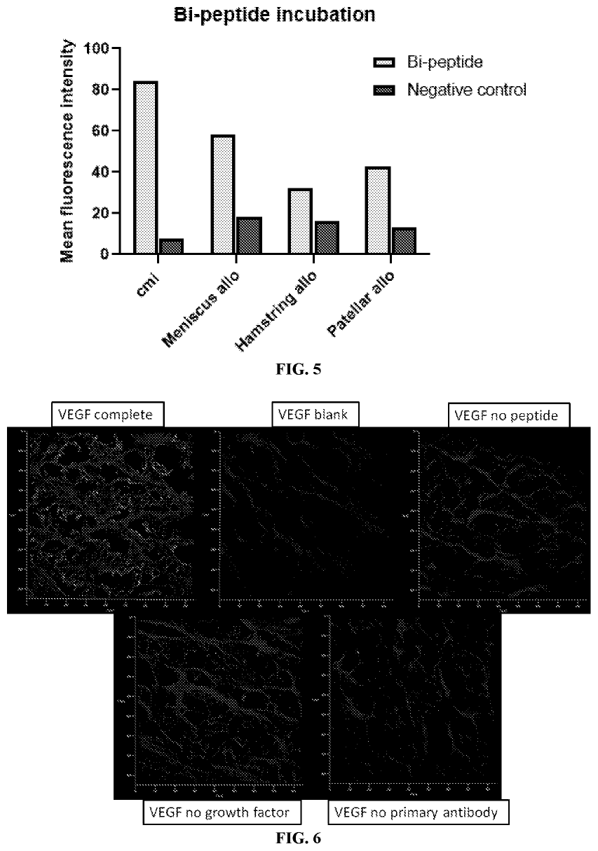

FIG. 5 is a graph that shows mean fluorescence intensity for each collagen-containing tissue after incubation with the fluorescently labelled collagen binding motif peptide.

### Example 2. Confirmation of VEGF and PDGF Capturing on the Collagen Meniscal Implant

Peptide sequence:
(SEQ ID NO: 5)
GLRSKSKKFRRPDIQYPDATDEDITSHM-
two 6 amino-hexanoic acid spacers-
sequence
(SEQ ID N: 25)
RLVFALGTDGKKLRIKSKEKCNDGK,
N-terminus:
Free NH2, C-terminus: Acid (COOH).

FIG. 6 shows images of an immunofluorescence assay for VEGF 125-a captured by the bi-peptide structure on the CMI. Pictures are merged z-stack files from confocal microscopy.

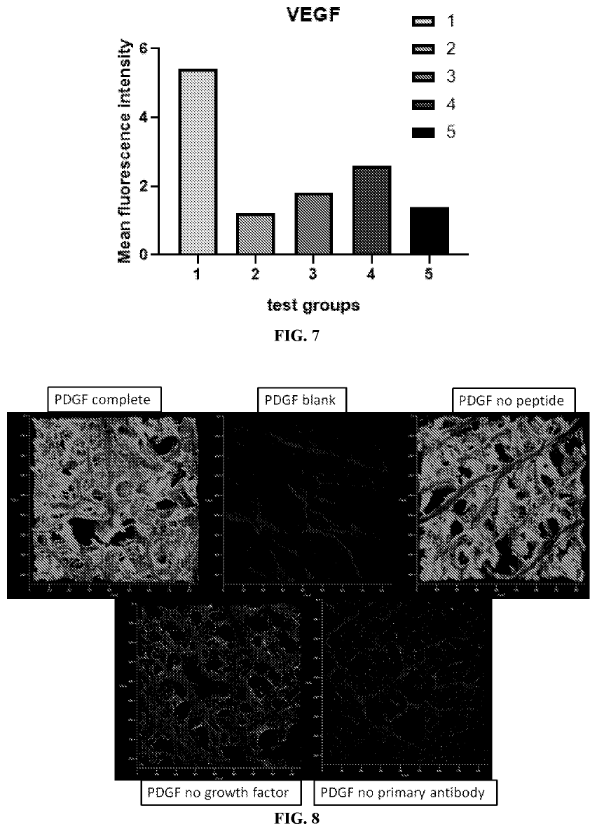

FIG. 7 is a graph that shows mean fluorescence intensity for the IF assay capturing VEGF. (1) Complete assay; (2) Blank assay; (3) Assay without peptide; (4) Assay without growth factor (VEGF); and (5) Assay without primary antibody.

FIG. 8 shows images of an immunofluorescence assay for PDGF-BB captured by the bi-peptide structure on the CMI. Pictures are merged z-stack files from confocal microscopy.

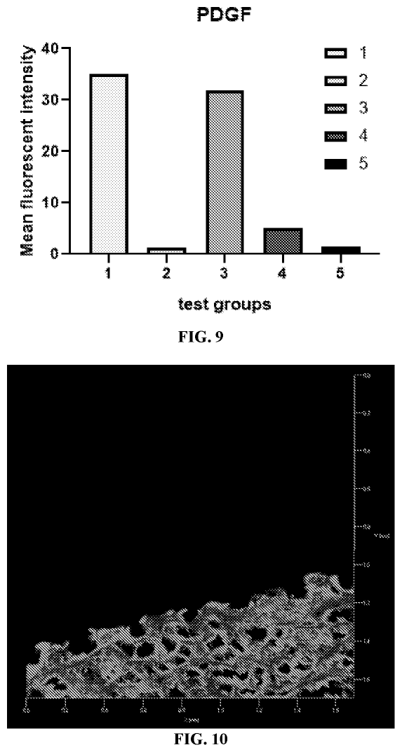

FIG. 9 is a graph that shows mean fluorescence intensity for the IF assay capturing PDGF. (1) Complete assay; (2) Blank assay; (3) Assay without peptide; (4) Assay without growth factor (PDGF); (5) Assay without primary antibody. Note: PDGF appears to bind well to collagen type 1 of the scaffold even without pre-incubation of the bi-peptide structure. Therefore the heparin-binding domain part of the peptide for PDGF can't be evaluated according to this assay.

FIG. 10 is an image of a Tile confocal scan of the CMI in the IF assay for PDGF-BB. The complete surface of the CMI was able to bind with recombinant PDGF.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Somasundaram et al. Type I, II, III, IV, V, and VI Collagens Serve as Extracellular Ligands for the Isoforms of Platelet-derived Growth Factor (AA, BB, and AB), 1998, The journal of biological chemistry.

1. A bi-peptide comprising a formula:
peptide 1ₙ-linkerₙ-peptide 2ₙ, or
peptide 2ₙ-linkerₙ-peptide 1ₙ
wherein peptide 1 has an affinity to a growth factor or a growth factor receptor,
wherein peptide 2 has an affinity for an extracellular matrix protein, and
wherein the linker is a chemical or peptide linker; and
n is one or more.

2. The bi-peptide of claim 1, wherein peptide 1 is selected from a TGF-B1, VEGF, BMP-2, PDGF, or an FGF binding peptide: peptide 2 is selected from a collagen, fibronectin, hyaluronan, hydroxyapatite, or a heparin binding peptide; or both.

3. (canceled)

4. The bi-peptide of claim 1, wherein peptide 1 is selected from LPLGNSH (SEQ ID NO:1), SWWAPFH (SEQ ID NO:2), YPVHPST (SEQ ID NO:3), or a PILQAGL (SEQ ID NO:4) peptide.

5. The bi-peptide of claim 1, wherein peptide 2 is selected from
(SEQ ID NO: 5)
GLRSKSKKFRRPDIQYPDATDEDITSHM,
(SEQ ID NO: 6)
FNKHTEIIEEDTNKDKPSYQFGGHNSVDFEEDTLPKV,
(SEQ ID NO: 7)
GAHWQFNALTVR,
(SEQ ID NO: 8)
GKKQRFRHRNRKG,
(SEQ ID NO: 9)
NNHYLPR,
or
(SEQ ID NO: 25)
RLVFALGTDGKKLRIKSKEKCNDGK
peptide.

6. The bi-peptide of claim 1, further comprising at least one of:
binding the bi-peptide to a polymer;
binding a second linker attached to at least one of peptide 1, peptide 2, or both, opposite the linker, and one or more additional peptide 1, peptide, or both peptide 1 and peptide 2 attached to the second linker;
forming concatamers of peptide 1, peptide 2, or both attached to peptide 1, peptide 2, or both; or
the linker is selected from a small molecule, a peptide, a nucleic acid, a carbohydrate, or a lipid.

7. (canceled)

8. (canceled)

9. (canceled)

10. A device comprising the bi-peptide of claim 1.

11. The bi-peptide of claim 1, provided in an amount sufficient to treat a tissue pathology or tissue engineering, preferably a tissue injury.

12. The bi-peptide of claim 1 provided in an amount sufficient to treat an injured tendon and/or ligament.

13. A method for treatment of a tissue pathology in a subject, the method comprising
(a) providing a bi-peptide comprising a formula:
peptide 1ₙ-linkerₙ-peptide 2ₙ, or
peptide 2ₙ-linkerₙ-peptide 1ₙ
wherein peptide 1 has an affinity to a growth factor or a growth factor receptor,
wherein peptide 2 has an affinity for an extracellular matrix protein,
wherein the linker is a chemical or peptide linker, and
n is one or more;
(b) introducing the bi-peptide or device into tissue of the subject; and
(c) allowing the bi-peptide or device to capture growth factor from the subject.

14. The method of claim 13, further comprising mixing or attaching the bi-peptide to a biopolymer, wherein the biopolymer is selected from the group consisting of collagen, chitosan, dextran, hyaluronic acid, heparin, polysaccharides such as alginate, hyaluronic acid and agarose, polynucleotides, polypeptides, starch, polylactic acid, poly-L-lactic acid, polyglycolic acid, polyglycolic lactic acid, poly(amidoamine), poly(caprolactone), polyalkyleneoxide-polyalkylene-terephtalate block copolymer, poly-N-isopropylacrylamide, polyurethane, poly-acrylate, polyesters, polystyrene, polycarbonate, polyethyleneterephtalate (PET) polybutyleneterephtalate (PBT), polyethyleneoxide (PEO), polyethersulfone (PES), polytetrafluoroethylen (PTFE), polytrimethylenecaprolactone (PTMC), polyanhydride, poly(ortho)ester, polyphosphazene, and/or combinations thereof.

15. The method of claim 13, wherein the tissue injury is an injured tendon and/or ligament.

16. The method of claim 13, wherein peptide 1 is selected from a TGF-B1, VEGF, BMP-2, or an FGF binding peptide; or peptide 2 is selected from a collagen, fibronectin, hyaluronan, hydroxyapatite or a heparin binding peptide; or both.

17. (canceled)

18. The method of claim 13, wherein peptide 1 is selected from LPLGNSH (SEQ ID NO:1), SWWAPFH (SEQ ID NO:2), YPVHPST (SEQ ID NO:3), or a PILQAGL (SEQ ID NO:4) peptide.

19. The method of claim 13, wherein peptide 2 is selected from
(SEQ ID NO: 5)
GLRSKSKKFRRPDIQYPDATDEDITSHM,
(SEQ ID NO: 6)
FNKHTEIIEEDTNKDKPSYQFGGHNSVDFEEDTLPKV,
(SEQ ID NO: 7)
GAHWQFNALTVR,
(SEQ ID NO: 8)
GKKQRFRHRNRKG,
(SEQ ID NO: 9)
NNHYLPR,
or
(SEQ ID NO: 25)
RLVFALGTDGKKLRIKSKEKCNDGK
peptide.

20. The method of claim 13, further comprising at least one of:
binding the bi-peptide to a polymer;
binding a second linker attached to at least one of peptide 1, peptide 2, or both, opposite the linker, and one or more additional peptide 1, peptide, or both peptide 1 and peptide 2 attached to the second linker; or
forming concatamers of peptide 1, peptide 2, or both attached to peptide 1, peptide 2, or both.

21. (canceled)

22. (canceled)

23. The method of claim 13, wherein the linker is selected from a small molecule, a peptide, a nucleic acid, a carbohydrate, or a lipid.

24. A method of making a bi-peptide comprising a formula:
peptide 1ₙ-linkerₙ-peptide 2ₙ, or
peptide 2ₙ-linkerₙ-peptide 1ₙ
obtaining a peptide 1 that has an affinity to a growth factor or a growth factor receptor;
connecting a linker to peptide 1, wherein the linker is a chemical or peptide 1; and
connecting a peptide 2 has an affinity for an extracellular matrix protein to the linker, wherein n is one or more.

25. The method of claim 24, further comprising selecting peptide 1 from a TGF-B1, VEGF, BMP-2, or an FGF binding peptide; selecting peptide 2 from a collagen, fibronectin, hyaluronan, hydroxyapatite, or a heparin binding peptide; or both.

26. (canceled)

27. The method of claim 24, further comprising selecting peptide 1 from LPLGNSH (SEQ ID NO:1), SWWAPFH (SEQ ID NO:2), YPVHPST (SEQ ID NO:3), or a PILQAGL (SEQ ID NO:4) peptide.

28. The method of claim 24, further comprising selecting peptide 2 from
(SEQ ID NO: 5)
GLRSKSKKFRRPDIQYPDATDEDITSHM,
(SEQ ID NO: 6)
FNKHTEIIEEDTNKDKPSYQFGGHNSVDFEEDTLPKV,
(SEQ ID NO: 7)
GAHWQFNALTVR,
(SEQ ID NO: 8)
GKKQRFRHRNRKG,
(SEQ ID NO: 9)
NNHYLPR,
or
(SEQ ID NO: 25)
RLVFALGTDGKKLRIKSKEKCNDGK
peptide.

29. The method of claim 24, further comprising at least one of:
binding the bi-peptide to a polymer;
binding a second linker attached to at least one of peptide 1, peptide 2, or both, opposite the linker, and one or more additional peptide 1, peptide, or both peptide 1 and peptide 2 attached to the second linker; or
forming concatamers of peptide 1, peptide 2, or both attached to peptide 1, peptide 2, or both.

30. (canceled)
